(19) United States
(12) Patent Application Publication
(10) Pub. No.: US 2024/0024287 A1
(43) Pub. Date: Jan. 25, 2024

(54) APPLICATION OF ANLE138B IN DRUG FOR IMPROVING DIET-INDUCED INSULIN RESISTANCE

(71) Applicant: The First Affiliated Hospital of Shantou University Medical College, Shantou (CN)

(72) Inventor: Jie WU, Shantou (CN)

(73) Assignee: The First Affiliated Hospital of Shantou University Medical College, Shantou (CN)

(21) Appl. No.: 17/975,623

(22) Filed: Oct. 28, 2022

(30) Foreign Application Priority Data
Jul. 22, 2022 (CN) 202210866348.3

(51) Int. Cl. A61K 31/4155; A61P 3/08

(52) U.S. Cl. CPC A61K 31/4155; A61P 3/08

(57) ABSTRACT

An application of Anle138b in a drug for improving diet-induced insulin resistance is provided. Anle138b has obvious improvement effects on both preventing and treating insulin resistance, and can reduce the intake efficiency and the body weight. Besides, Anle138b can effectively improve diet-induced ectopic fat storage and adipose tissue hypertrophy, and the effect of Anle138b on improving insulin resistance is independent of the reduction of food intake.

### CROSS REFERENCE TO THE RELATED APPLICATIONS

This application is based upon and claims priority to Chinese Patent Application No: 202210866348.3, filed on Jul. 22, 2022, the entire contents of which are incorporated herein by reference.

### TECHNICAL FIELD

The present invention relates to the technical field of insulin resistance, and in particular to an application of Anle138b in a drug for improving diet-induced insulin resistance.

### BACKGROUND

As a core concept of metabolic syndromes, insulin resistance accompanies with the occurrence and development of the entire course of metabolic diseases, such as diabetes and obesity, as well as the occurrence and development of cardiovascular and other complications. In recent years, studies have found that in addition to peripheral insulin resistance, the pathogenic role of central insulin resistance has gradually become more important. Since insulin, insulin receptors and their signaling pathways are widely distributed in brains, especially dominated by the distribution in the hypothalamus for energy metabolism regulation, in the central nervous system, in addition to regulating the expression of neuropeptides and participating in the production of hepatic glucose, insulin, insulin receptors and their signaling pathways also play important roles in the normal metabolism of neurons and the function of tactile transmission, and it has been found that insulin resistance accompanies with a variety of neuronal dysfunction diseases. Several epidemiological studies have shown that insulin resistance increases the risk of dementia and AD. Nutrition-induced insulin resistance significantly affects neural insulin signaling and leads to cognitive impairment. Because insulin resistance is closely related to functions of the nervous system, more scholars now refer to AD as type 3 diabetes mellitus.

Anle138b, as a highly active low molecular weight compound of 3,5-diphenyl-pyrazole (DPP) derivatives, has high blood-brain barrier permeability, has significant curative effects on regulating intracranial amyloid deposition in both prevention and treatment, can directly interfere with the pathological aggregation of amyloid proteins such as prion, α-synuclein and Tau protein, and can improve the pathological progression of animal models of neurodegenerative diseases, such as multiple system atrophy, Parkinson's disease, and Alzheimer's disease.

### SUMMARY

The present invention is intended to provide an application of Anle138b in a drug for improving diet-induced insulin resistance. Anle138b has obvious ameliorative effects on both preventing and treating insulin resistance, and can reduce the intake efficiency and the body weight. Besides, Anle138b can effectively improve diet-induced ectopic fat storage and adipose tissue hypertrophy, and the effect of Anle138b on improving the insulin resistance is independent of the reduction of food intake.

In order to achieve the above objective, the present invention provides an application of Anle138b in a drug for improving diet-induced insulin resistance.

Preferably, improvement of insulin resistance includes prevention or treatment for improvement of insulin resistance.

An application of Anle138b in preparing a drug for reducing the food intake and body weight of patients with insulin resistance diseases.

Preferably, the insulin resistance is ameliorated by reducing the high-fat feeding efficiency and restoring insulin reactivity.

An application of Anle138b in preparing a drug for alleviating hepatic and pancreatic steatosis induced by insulin resistance.

Preferably, alleviating hepatic and pancreatic steatosis induced by insulin resistance is unrelated to reducing the food intake.

An application of Anle138b in preparing a drug for reducing the blood lipid of patients with insulin resistance diseases.

An application of Anle138b in preparing a drug for suppressing appetite in the process of losing weight.

Therefore, the application of Anle138b in a drug for improving diet-induced insulin resistance is adopted in the present invention, and it is found by judging the therapeutic effect of Anle138b on the high-fructose-induced rat insulin resistance models that the intraperitoneal injection of Anle138b 1 mg/kg for 2 weeks can improve the hyperinsulinemia of the rats and reverse the high-fructose-induced insulin resistance of the rats.

The technical solutions of the present invention will be further described in detail below in combination with the drawings and the embodiments.

### BRIEF DESCRIPTION OF THE DRAWINGS

FIG. 1 shows body weight growth curves of the control group and the model group.

FIGS. 2A-2B show changes in the body weight of the high-fructose-diet-induced rat insulin resistance model after Anle138b intervention; wherein FIG. 2A shows the comparison of the body weight among the normal control group, the normal drug group and the high-fructose model control group before and after intervention; and FIG. 2B shows the comparison of the body weight of the rat insulin resistance model before and after intervention with different concentrations of Anle138b, where L-THP is the positive control.

FIGS. 3A-3D show changes in the metabolic indexes of the rats after intervention with different concentrations of Anle138b; wherein FIG. 3A shows the comparison of fasting blood glucose values among the normal control group, the model control group, the positive control group and after intervention with Anle138b 3 mg/kg; FIG. 3B shows the comparison of fasting blood glucose values after intervention with different concentrations of Anle138b; FIG. 3C shows the comparison of fasting serum insulin concentrations among the normal control group, the model control group, the positive control group and after intervention with Anle138b 3 mg/kg; and FIG. 3D shows the comparison of fasting serum insulin concentrations after intervention with different concentrations of Anle138b.

FIGS. 4A-4B show changes in the insulin resistance values of the rats after administration with different concentrations of Anle138b; wherein FIG. 4A shows the comparison of the insulin resistance values among the normal control group, the model control group, the positive control group and after intervention with Anle138b 3 mg/kg; and FIG. 4B shows the comparison of the insulin resistance values after intervention with different concentrations of Anle138b.

FIGS. 5A-5B shows changes in the glucose tolerance of the rats after administration with different concentrations of Anle138b; wherein FIG. 5A shows the changes in the glucose tolerance of the rats after administration with different concentrations of Anle138b; and FIG. 5B shows the comparison of the areas under curve (AUC) of glucose tolerance.

FIG. 6 shows the comparison of the epididymal fat weight index of the rats after administration with different concentrations of Anle138b.

FIG. 7 shows the organic pathomorphism—HE staining (×400) of the rats after administration with different concentrations of Anle138b.

FIGS. 8A-8C show changes in the food intake and body weight of the high-fat-induced rat insulin resistance model after Anle138b intervention; wherein FIG. 8A shows the change curve of the food intake among the normal control group, the model control group and the Anle138b (different-concentration) drug groups; FIG. 8B shows the change curve of the body weight among the normal control group, the model control group and the Anle138b (different-concentration) drug groups; and FIG. 8C shows the comparison of the feeding efficiency between the model control group and the Anle138b (different-concentration) drug control groups.

FIG. 9 shows changes in the metabolic indexes of the high-fat-induced rat insulin resistance model after Anle138b intervention; wherein FIG. A shows the comparison of the fasting blood glucose between the high-fat model control group and the Anle138b (different-concentration) drug groups; FIG. B shows the comparison of the fasting triglyceride between the high-fat model control group and the Anle138b (different-concentration) drug groups; FIG. C shows the comparison of the fasting serum insulin between the high-fat model control group and the Anle138b (different-concentration) drug groups; and FIG. D shows the comparison of the insulin resistance index between the high-fat model control group and the Anle138b (different-concentration) drug groups.

FIGS. 10A-10B show changes in the glucose tolerance of the high-fat-induced rat insulin resistance model after Anle138b intervention; wherein FIG. 10A shows the curves of the glucose tolerance of the model control group and the Anle138b (different-concentration) drug groups; and FIG. 10B shows the comparison of the areas under curve (AUC) of the glucose tolerance between the model control group and the Anle138b (different-concentration) drug groups.

FIGS. 11A-11B show changes in the liver weight index of the high-fat-induced rat insulin resistance model after Anle138b intervention; wherein FIG. 11A shows the comparison of the liver weight index between the model control group and the Anle138b (different-concentration) drug groups; and FIG. 11B shows the comparison of the fat weight index between the model control group and the Anle138b (different-concentration) drug groups.

FIG. 12 shows the organic pathomorphism—HE staining (×400) of the rats after administration with different concentrations of Anle138b.

FIGS. 13A-13C show changes in the food intake and body weight of the high-fat-induced rat insulin resistance model after prevention by Anle138b; wherein FIG. 13A shows the change curve of body weight of the model control group and the Anle138b 5 mg/kg drug group; FIG. 13B shows the change curve of food intake of the model control group and the Anle138b 5 mg/kg drug group; and FIG. 13C shows the comparison of the feeding efficiency between the model control group and the Anle138b 5 mg/kg drug group.

FIGS. 14A-14E show changes in the metabolic indexes of the high-fat-induced rat insulin resistance model after prevention by Anle138b; wherein FIG. 14A shows the comparison of the fasting blood glucose between the model control group and the Anle138b 5 mg/kg drug group; FIG. 14B shows the comparison of the fasting triglyceride between the model control group and the Anle138b 5 mg/kg drug group; FIG. 14C shows the comparison of the total serum cholesterol between the model control group and the Anle138b 5 mg/kg drug group; FIG. 14D shows the comparison of the fasting serum insulin between the model control group and the Anle138b 5 mg/kg drug group; and FIG. 14E shows the comparison of the insulin resistance index between the model control group and the Anle138b 5 mg/kg drug group.

FIGS. 15A-15B show changes in the glucose tolerance of the high-fat-induced rat insulin resistance model after prevention by Anle138b; wherein FIG. 15A shows the curves of glucose tolerance of the model control group and the Anle138b 5 mg/kg drug group; and FIG. 15B shows the comparison of the areas under curve (AUC) of glucose tolerance between the model control group and the Anle138b 5 mg/kg drug group.

FIGS. 16A-16B show changes in the liver weight index of the high-fat-induced rat insulin resistance model after prevention by Anle138b; wherein FIG. 16A shows the comparison of the liver weight index between the model control group and the Anle138b 5 mg/kg drug group; and FIG. 16B shows the comparison of the fat weight index between the model control group and the Anle138b 5 mg/kg drug group.

FIG. 17 shows the influence of the preventive administration with Anle138b 5 mg/kg on the organic pathomorphism—HE staining (×400) of the rat insulin resistance model.

FIGS. 18A-18B show changes in the body weight and feeding efficiency of the rat insulin resistance model in the Anle138b treatment group and the PF group; wherein FIG. 18A shows the change curve of body weight of the Anle138b 5 mg/kg drug group and the PF group; and FIG. 18B shows the comparison of the feeding efficiency between the Anle138b 5 mg/kg drug group and the PF group.

FIGS. 19A-19B show changes in the metabolic indexes of the rat insulin resistance model in the Anle138b treatment group and the PF group; wherein FIG. 19A shows the comparison of the fasting blood glucose between the Anle138b 5 mg/kg drug group and the PF group; and FIG. 19B shows the comparison of the serum triglyceride between the Anle138b 5 mg/kg drug group and the PF group.

FIG. 20 shows the comparison of the rat insulin resistance value between the Anle138b treatment group and the PF group.

FIGS. 21A-21B show changes in the glucose tolerance of the rat insulin resistance model in the Anle138b treatment group and the PF group; wherein FIG. 21A shows the glucose tolerance curves of the Anle138b 5 mg/kg drug group and the PF group; and FIG. 21B shows the comparison of the AUC of glucose tolerance between the Anle138b mg/kg drug group and the PF group.

FIGS. 22A-22B show changes in the liver weight index of the rat insulin resistance model in the Anle138b treatment group and the PF group; wherein FIG. 22A shows the comparison of the liver weight index between the Anle138b 5 mg/kg drug group and the PF group; and FIG. 22B shows the comparison of the epididymal fat weight index between the Anle138b 5 mg/kg drug group and the PF group.

FIG. 23 shows the influence on the organic pathomorphism—HE staining (×400) of the rat insulin resistance model in the Anle138b 5 mg/kg drug group and the PF group.

### DETAILED DESCRIPTION OF THE EMBODIMENTS

The technical solutions of the present invention will be further described below in combination with the drawings and the embodiments.

Unless defined otherwise, all terms (including technical or scientific terms) used in the present disclosure have the same meaning as commonly understood by those of ordinary skill in the art to which the present disclosure belongs. It should also be understood that the terms defined in general-purpose dictionaries should be construed as having meanings consistent with their meanings in the context of the related art, and should not be interpreted in an idealized or highly formalized sense, unless explicitly defined in such a way in this context.

For those skilled in the art, it is obvious that the present invention is not limited to the details of the above exemplary embodiment, and the present invention can be implemented in other specific forms under the situation of not deviating from the main idea or basic features of the present invention. Therefore, from any point of view, the embodiment shall be regarded as exemplary and non-limiting. The scope of the present invention is defined by the attached claims rather than the above description.

8-week-old male C57BL/6J rats were selected as experimental animals. The rats were adaptively fed for 1 week after purchase, and then weighed for the fasting body weight. The healthy rats weighing 22-24 g were selected and randomly divided into groups according to the experimental flow design. (1) The rats were fed with normal feed and high-fructose feed for 8 weeks; (2) the rats were fed with high-fat feed for 12 weeks; (3) the rats were fed with high-fat feed for 8 weeks as a preventive experiment. Fasting body weights were measured and recorded regularly every week, and the rats were guaranteed to have free access to water and food during feeding. The food intake, water intake, and free movement of the model rats were continuously observed, and the rats with stunted growth, rough hair, and rickety posture after eating high-fructose and high-fat diets were screened out. In the experiment later, the rats were fed with normal feed or high-fructose/high-fat feed during the 2 weeks of intraperitoneal injection to ensure the consistency before and after administration.

Formula of high-fructose feed: the high-fructose feed had a fructose calorie of 66.7%, a carbohydrate calorie of 66.9%, a fat calorie of 12.9% and a protein calorie of 20.2%, with a total calorie of 3.6 kcal/g, and the high-fructose feed was provided by Jiangsu Medison Biomedical Co., Ltd.

High-fat formula: the high-fat purified feed had a fat energy supply ratio of 45%, wherein the energy ratio of protein was 20% and the energy ratio of carbohydrate was 35%. A total energy-to-mass ratio was 4.73 kcal/g. The feed was provided by Beijing Huafukang Biotechnology Co., Ltd.

Anle138b solutions: The drug doses required for 1 week of intervention for each group of rats were calculated, Anle138b drug concentrations of 1 mg/kg (0.01%), 3 mg/kg (0.03%) and 5 mg/kg (0.05%) were prepared, Anle138b powder was weighed and then added in 10% DMSO, 40% PEG300, 5% Tween-80 and 45% saline in sequence, each mixture was evenly blended by vortex into a clear solution, and each Anle138b solution was separately bottled according to the daily dose and stored in a refrigerator at 4° C.

L-THP solution: The drug dose required for 1 week of intervention for each group of rats was calculated, L-THP drug concentrations of 3 mg/kg (0.03%) was prepared, light yellow L-THP powder was weighed and then added in 10% DMSO, 40% PEG300, 5% Tween-80 and 45% saline in sequence, each mixture was evenly blended by vortex into a clear solution, and each Anle138b solution was separately bottled according to the daily dosage and stored in a refrigerator at 4° C.

The 8-week-old adult rats were modeled by changing the diets after 1 week of adaptive feeding. Blood was collected from the right orbital venous plexus with a glass tube at 8 weeks and 12 weeks after model establishment and after fasting (>12 h), and blood glucose levels were measured with a blood glucose tester (Sannuo GA-3); and the insulin resistance mode was judged by the subsequent determination of metabolic indexes, such as glucose tolerance test by intraperitoneal injection, fasting serum insulin (enzyme-linked immunosorbent assay, Raybiotech), serum triglyceride (GPO-PAP single-reagent microplate method, Nanjing Jiancheng) and cholesterol (GPO-PAP single-reagent microplate method, Nanjing Jiancheng).

### EXAMPLE 1

### Influence of Anle138b on High-Fructose-Induced Rat Insulin Resistance Models

(1) The normal rats were randomly divided into two groups: blank control group (n=6) and drug control group (n=7); the rats in the normal control group were intraperitoneally injected with a similar volume of solvent and Anle138b (3 mg/kg, 0.03%) according to the body weight for 2 weeks.

The rats were fed with 60% high-fructose diet for 8 weeks to establish insulin resistance models, and the rats fed with normal diet provided by the animal room of Shantou University Medical College were used as controls. After fasting, orbital blood was collected, and fasting blood glucose, serum insulin, glucose tolerance were determined to judge whether the modeling was successful. According to an insulin resistance index (HOMA-IR) as the core judgment index, the successfully modeled rats were enrolled by screening the IR index of >3 and randomly divided into groups to explore the influence of different concentrations of Anle138b on the rat insulin resistance models. The concentration gradients for different groups were 0 mg/kg (injection solvent, namely the model control group), 1 mg/kg, 3 mg/kg and 5 mg/kg clear Anle138b solution, as well as the L-THP 3 mg/kg positive drug control group. The rats were administrated by intraperitoneal injection for 2 weeks continuously, once a day.

Anle138b 1 mg/kg, 3 mg/kg and 5 mg/kg could all effectively reduce the fasting blood glucose, improve hyperinsulinemia, and reduce the insulin resistance value. In addition to reducing the epididymal fat weight index, Anle138b 1 mg/kg, 3 mg/kg and 5 mg/kg pathologically could also effectively improve the liver steatosis and epididymal adipose tissue hyperplasia caused by high-fructose diet, and could alleviate the inflammatory response of tissues. This indicated that Anle138b had a therapeutic effect on the high-fructose-induced insulin resistance of rats.

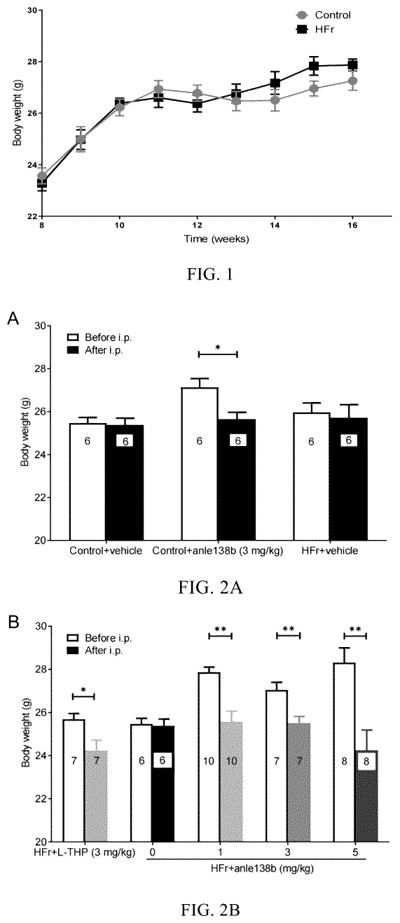

The body weights of the IR model group and the normal control group during modeling are as shown in Table 1, and the development trend is as shown in FIG. 1. There was no significant difference in the average body weight between the model group and the control group, and the overall growth trend was similar, wherein the body weight of the high-fructose model group increased at Week 14, Week 15 and Week 16, but there was no statistical significance for the difference.

TABLE 1
Comparison of Changes in Body Weights (g) from Week 8 to Week 16
between the Control Group and the Model Group

| | Model Group | Control group |
|---|---|---|
| Week 8 | 23.28 ± 0.92 | 23.57 ± 0.93 |
| Week 9 | 24.97 ± 1.21 | 24.99 ± 1.47 |
| Week 10 | 26.52 ± 0.85 | 26.22 ± 0.97 |
| Week 11 | 26.6 ± 1.21 | 26.93 ± 1.01 |
| Week 12 | 26.37 ± 1.02 | 26.78 ± 0.95 |
| Week 13 | 26.76 ± 1.16 | 26.47 ± 1.12 |
| Week 14 | 27.17 ± 1.40 | 26.50 ± 1.25 |
| Week 15 | 27.83 ± 1.13 | 26.96 ± 0.88 |
| Week 16 | 27.87 ± 0.74 | 27.26 ± 1.11 |

### (2) Influence of Different Concentrations of Anle138b on the Body Weights of High-Fructose-Induced Rat Insulin Resistance Models

In the self-control experiment of the normal control group before and after administration, there was no statistical significance for the difference in the body weight of the control group before the solvent was administered and after 2 weeks from the intraperitoneal injection of the solvent. The body weight after administration of the Anle138b 3 mg/kg drug decreased as compared with the body weight before administration, and there was statistical significance for the difference. This indicated that the Anle138b drug had an effect on reducing the body weight of normal rats.

There was no statistical significance for the difference between the body weights before and after administration of the solvent in the high-fructose model group. The efficacy of the drug on the high-fructose model group was determined after excluding the influence of the intraperitoneal injection of the solvent on the body weight of the rats.

As shown in FIGS. 2A-2B, the body weight decreased after 2 weeks from the intraperitoneal injection of Anle138b 1 mg/kg, while the body weight decreased after intervention with high-dose Anle138b 3 mg/kg and 5 mg/kg; there was statistical significance for the differences. It could be seen that the Anle138b intervention had the effect of reducing body weights for both the normal rats and the model rats.

TABLE 2
Comparison of Changes in Body Weights (g) Before and After Drug
Intervention

| | | Body Weight Before | Body Weight After |
|---|---|---|---|
| | n | Administration (g) | Administration (g) |
| Normal Control | 6 | 25.47 ± 0.6377 | 25.38 ± 0.7600 |
| Group | | | |
| Normal Drug Group | 6 | 27.13 ± 0.9993 | 25.65 ± 0.7842* |
| Model Control | 6 | 25.97 ± 1.069 | 25.72 ± 1.485 |
| Group | | | |
| L-THP Positive | 7 | 25.69 ± 0.7058 | 24.24 ± 1.255* |
| Control Group | | | |
| Anle138b 1 mg/kg | 10 | 27.87 ± 0.7424 | 25.58 ± 1.555* * |
| Anle138b 3 mg/kg | 7 | 27.04 ± 0.9431 | 25.51 ± 0.8009** |
| Anle138b 5 mg/kg | 8 | 28.31 ± 1.927 | 24.25 ± +2.652** |

Note:
Compared with the body weight before administration, *P < 0.05, **P < 0.01.

### (3) Influence of Different Concentrations of Anle138b on the Metabolic Indexes of High-Fructose-Induced Rat Insulin Resistance Models

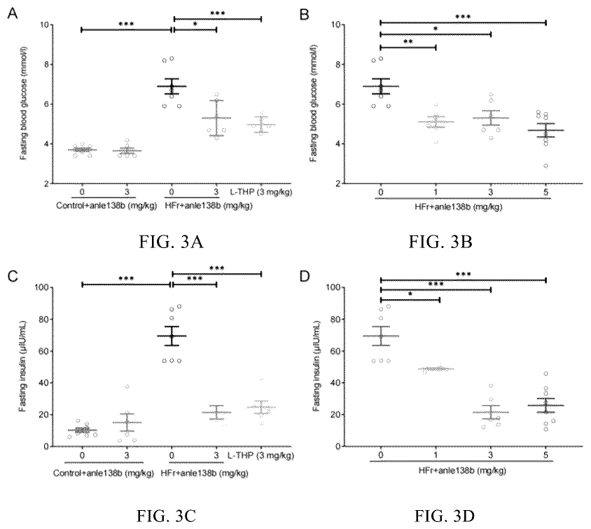

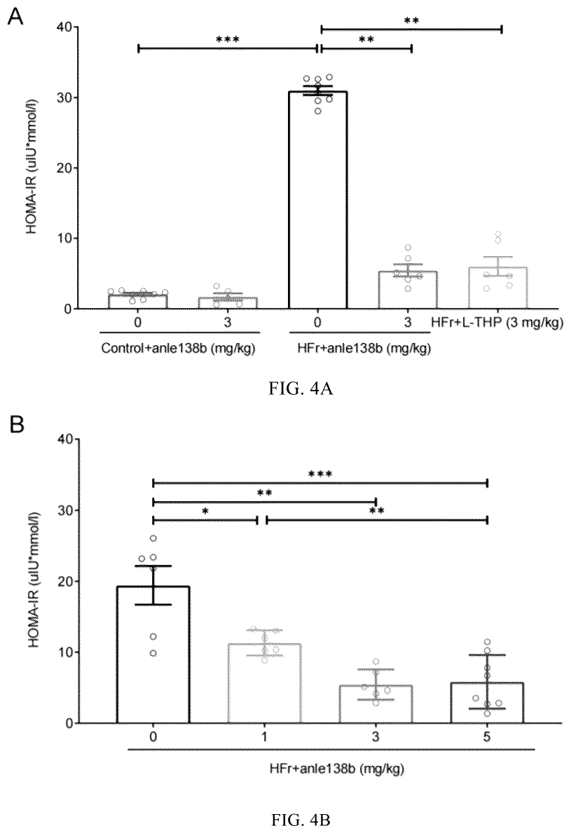

The influence of different concentrations of Anle138b on the metabolic indexes of the high-fructose-induced rat insulin resistance models is as shown in Table 3. High fructose treatment for 2 weeks could increase the fasting blood glucose levels of the rats (as shown in FIGS. 3A-3D).

For the rats treated with high fructose for 2 weeks, the fasting blood glucose levels of the Anle138b drug treatment group after intervention at 1 mg/kg, 3 mg/kg and 5 mg/kg were all lower than those of the model solvent control group. Therefore, it could be known that the Anle138b drug could effectively reduce the elevated blood glucose in the diet-induced model of insulin resistance. The fasting blood glucose level of the drug control group (normal rats) treated with Anle138b 3 mg/kg for 2 weeks was not significantly different from that of the blank control group, so Anle138b could only reduce the blood glucose of the rat insulin resistance models but had no obvious influence on the blood glucose level of the normal rats. The blood glucose of the positive control group after intervention with L-THP 3 mg/kg was significantly lower than that of the solvent control group, and there was statistical significance for the differences.

The changes in the serum blood lipids were measured after the models were fed for 8 weeks. The triglyceride value of the normal control group was (0.6733±0.6733) mmol/L (n=7); the triglyceride value of the high-fructose model group was (0.6827±0.2120) mmol/L (n=7); there was no statistical significance for the difference between the two groups (not shown in the figure).Considering that the high fructose feeding for 8 weeks failed to reach the level of increasing serum triglyceride, the elevated serum triglyceride could prolong the feeding time or serum blood lipid indicators which were more sensitive at the early period of high fructose feeding were measured, such as serum free fatty acids, latter liver triglyceride, etc., so changes in the blood lipids were not compared currently, and then feeding evaluation could be performed by changing high-fat feed to improve the time efficiency of feeding.

The fasting insulin value of the normal control group was (10.28±3.72) (n=8), the fasting insulin value after intervention with Anle138b 3 mg/kg for 2 weeks was (15.08±13.14) μIU/mL (n=6), and both were within a normal range. The fasting insulin value of the high-fructose model group was significantly higher than that of the normal group, and there was statistical significance for the difference (P<0.0001), indicating that feeding with 60% high-fructose diet for 8 weeks could lead to hyperinsulinemia of rats. Combined with the changes in the blood glucose, it was considered that the insulin resistance models were successfully established.

TABLE 3
Influence of Anle138b on Metabolic Indexes of High-fructose-induced
Rat insulin resistance models

| | | Fasting Blood | | |
|---|---|---|---|---|
| | | Glucose | Fasting Insulin | HOMA-IR |
| Group | n | mmol/L | μIU/mL | μIU/mL*mmol/L |
| Blank control | 8 | 3.700 ± 0.23 | 10.28 ± 3.72 | 2.055 ± 0.58 |

group

| | | | | |
|---|---|---|---|---|
| Drug Control | 6 | 3.650 ± 0.33 | 15.08 ± 13.14 | 1.1689 ± 1.15 |
| group | | | | |
| Model | 7 | 6.900 ± 0.99 | 69.52 ± 15.66* | 30.99 ± 1.77 |
| Control | | | | |
| Group | | | | |
| L-THP 3 | 6 | 4.967 ± 0.39 # # | 24.76 ± 9.70 # # # | 6.029 ± 3.33 # # |
| mg/kg | | # | | |
| Anle138b 1 | 6 | 5.100 ± 0.65 # # | 48.97 ± 1.38 # | 11.3 ± 1.78 # |
| mg/kg | | | | |
| Anle138b 3 | 6 | 5.300 ± 0.89 # | 21.46 ± 10.36 # # | 5.438 ± +2.13 # # |
| mg/kg | | | # | |
| Anle138b 5 | 8 | 4.675 ± 0.95 # # | 25.74 ± 12.24 # # | 5.827 ± 3.78 # # |
| mg/kg | | # | # | # |

Note:
compared with the blank control group, ***P < 0.001.
Compared with the model control group, # P < 0.05, # # P < 0.01, # # # P < 0.001.

The insulin resistance value was calculated in combination with the method for calculating the insulin resistance index (HOMA-IR) in the homeostasis model assessment method. The insulin resistance value of the normal control group was (2.055±0.58) μIU/mL*mmol/L, and that of the normal group after Anle138b intervention was (1.1689±1.153) μIU/mL*mmol/L, both were within a normal range, and there was no statistical significance for the difference. At the same time, the resistance value of the high-fructose model group was significantly higher than that of the normal group, and there was statistical significance for the difference (P<0.0001).

Comparing the insulin resistance values after intervention with different concentrations of Anle138b, the insulin resistance value after intervention with Anle138b 1 mg/kg was significantly lower than that of the model control group; the insulin resistance value after intervention with Anle138b 3 mg/kg was significantly lower than that of the model control group; the insulin resistance value after intervention with Anle138b 5 mg/kg was significantly lower than that of the model control group. The insulin resistance value of the positive control group after intervention with L-THP 3 mg/kg was lower than that of the model control group.

According to the comprehensive judgment in combination with the blood glucose, the insulin value and the insulin resistance value, Anle138b could effectively improve the high-fructose-induced insulin resistance of rats in a dose-dependent manner. More indexes would be further judged for corroboration subsequently.

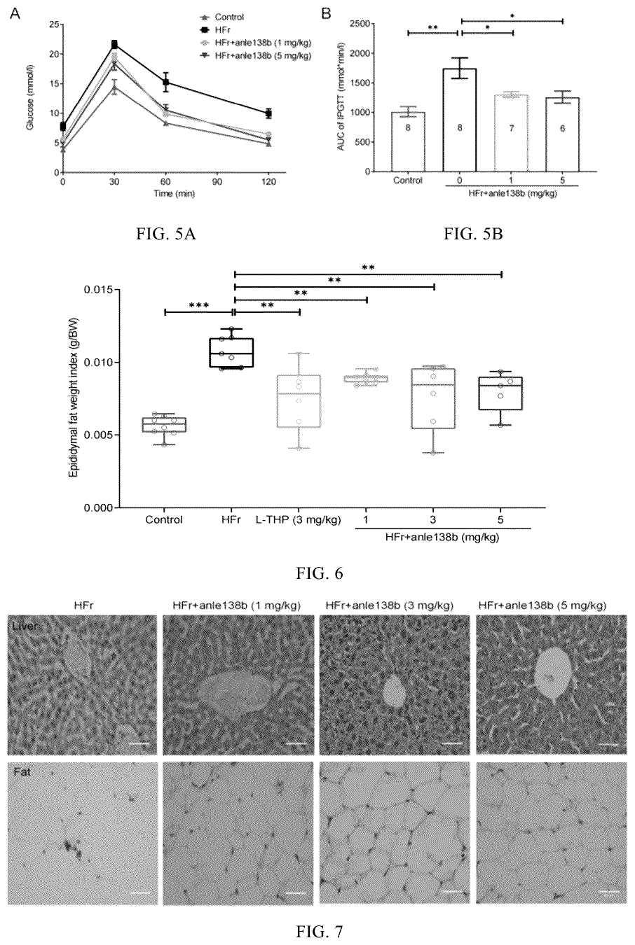

The results of the glucose tolerance test are as shown in FIGS. 5A-5B. Intervention with low-dose and high-dose Anle138b could both improve the glucose tolerance. The area under curve (AUC) was directly calculated by using the Graphpad Prism software, the AUC of the normal control group was (1,010±86.21) min*mmol/L (n=8), and the AUC value in the model group was (1,749±173.4) min*mmol/L (n=8), and the AUC value after intervention with Anle138b 1 mg/kg was (1,307±46.38) min*mmol/L (n=7), which was significantly lower than that of the model group with statistical significance for the difference (P<0.05); the AUC value after intervention with Anle138b 5 mg/kg was (1,261±103.2) min*mmol/L (n=6), which was significantly lower than that of the model group with statistical significance for the difference (P<0.05).

### (4) Influence of Different Concentrations of Anle138b on the Epididymal Fat Weight Index of High-Fructose-Induced Rat Insulin Resistance Models

The right epididymal fat weight index was calculated by the right epididymal fat weight/fasting body weight, and it was obtained that the fat weight index of the normal solvent control group was 0.005624±0.0007023 (n=8); the fat weight index of the high-fructose solvent group was 0.01082±0.001067 (n=7); compared with the normal solvent group, the fat weight per unit body weight was significantly increased, and there was statistical significance for the difference (P<0.001).

The fat weight index after intervention with Anle138b 1 mg/kg was 0.008918±0.0003788 (n=7), which was lower than that of the model solvent control group, P<0.01; the fat weight index after intervention with Anle138b 3 mg/kg was 0.007657±0.002366 (n=6), P<0.01; the fat weight index after intervention with Anle138b 5 mg/kg was 0.007971±0.001420 (n=5), P<0.01; the fat value was close with that of the L-THP positive control group (0.007510±0.002279, n=6), and as shown in FIG. 6, Anle138b intervention could effectively reduce the increase in the visceral fat weight induced by the high-fructose diet.

### (5) Influence of Anle138b on Changes in the Organic Pathomorphism of High-Fructose-Induced Rat Insulin Resistance Models

After 8 weeks of high-fructose feeding, the livers of the rats were pathologically examined; under a low-power microscope, the unclear demarcation of hepatic lobules of the rats could be observed; under a high-power microscope, the structural damage of the central hepatic vein, the disordered arrangement of hepatic cords, and the microcystic changes of fat accumulation in hepatocytes, namely hepatic steatosis, could be observed, the fusion of small lipid droplets and the death of hepatocytes could be partially observed; after intervention with Anle138b, it could be observed that the hepatocyte damage was alleviated in a dose-dependent manner, the hepatic steatosis was significantly alleviated, the central vein structure and the arrangement structure of the hepatic cord were restored, and the infiltration of inflammatory cells was reduced; this indicated that the drug application could effectively alleviate the hepatocyte injury induced by high fructose, as shown in FIG. 7, where the scale bar was 50 μm.

The pathological examination of the epididymal adipose tissue showed that in the high-fructose group the fat structure was disordered, the coronal structure formed by aggregated inflammatory cells was observed in the fat space, the fat cells were enlarged in different sizes and irregular shapes, and part of the cells were fused into big lipid droplets; after 2 weeks of drug treatment with Anle138b, it could be seen that with the increase of the drug concentration, the adipocytes were reduced in sizes and arranged regularly, and the tubular structure was reduced; the pathological examination of the adipose tissue showed that the drug reduced the nutritional burden of adipocytes by reducing the food intake to improve the early diet-induced fat inflammation and slow down the disease progression.

The pathological examination of pancreas after 8 weeks of high-fructose feeding showed no obvious structural damage of islets and acini.

### EXAMPLE 2

### Influence of Anle138b on High-Fat-Induced Rat Insulin Resistance Models

The rat IR models were randomly divided into two groups, the rats were administrated with Anle138b 3 mg/kg (n=6, 0.03%) and 5 mg/kg (n=6, 0.05%) for 2 weeks continuously, once a day.

Anle138b 3 mg/kg and 5 mg/kg could effectively reduce the food intake and body weight, reduce the feeding efficiency, reduce the fasting blood glucose and triglyceride, improve hyperinsulinemia, and reduce the insulin resistance value. Anle138b 3 mg/kg and 5 mg/kg could lower the liver weight index and the epididymal fat weight index. Pathologically, Anle138b 3 mg/kg and 5 mg/kg could significantly improve liver steatosis, pancreatic acinar steatosis, pathological hyperplasia of epididymal adipose tissue and intermuscular adipose hyperplasia caused by high fat, and reduce the inflammatory response of various tissues. This experiment showed that Anle138b had a therapeutic effect on the high-fat-induced insulin resistance of rats.

### (1) Influence of Anle138b on the Food Intake and Body Weight of High-Fat-Induced Rat Insulin Resistance Models

After the models were determined to be successful, the average value of food intake of each rat in the first two days was taken as its own food intake base. After the drug intervention, the daily food intake and body weight were measured and compared with their own bases for comparison of the standardized food intake and body weight of each rat.

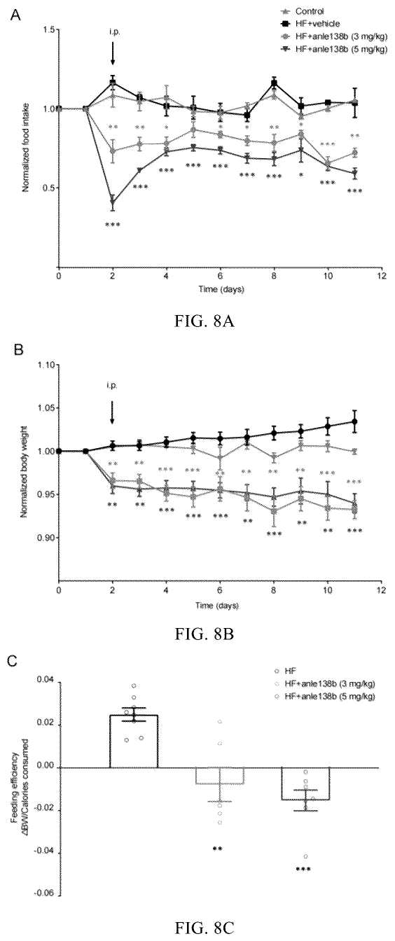

The average daily food intake of the high-fat fed rats was (2.789±0.3928) g/day (n=5); the average daily food intake of the Anle138b 3 mg/kg group was (1.946±0.4777) g/day (n=5), which was significantly lower than that of the model group, and there was statistical significance for the difference (P<0.001); the average daily food intake of the Anle138b 5 mg/kg group was significantly lower than that of the model group, and there was statistical significance for the difference (P<0.001). As shown in FIGS. 8A-8C, the daily food intake after drug intervention was compared with that of the model group, the food intake decreased after the drug intervention, and the decrease was more obvious at high doses.

For the high-fat fed rats, the weight before intervention was (31.01±1.332) g; the weight after intervention with Anle138b 3 mg/kg was (28.97±1.657) g (n=6), and the weight after intervention with Anle138b 5 mg/kg was (28.9±1.685) g (n=5); compared with the model group, the body weight was decreased, and there was statistical significance for the differences (P<0.05).The body weight decreased after the drug intervention, and the decrease was more obvious at high doses.

The feeding efficiency of each rat was obtained by the daily body weight change/energy consumption (g/calories/rat/day); the feeding efficiency of the high-fat feeding model group was 0.02491±0.008717; the feeding efficiency was −0.007788±0.01949 after intervention with Anle138b 3 mg/kg and −0.01528±0.01287 after intervention with Anle138b 5 mg/kg; compared with the model group, it could be seen that Anle138b could increase the feed utilization, reduce the consumption of high-fat feed, and reduce the feeding efficiency.

### (2) Influence of Anle138b on the Metabolic Indexes of High-Fat-Induced Rat Insulin Resistance Models

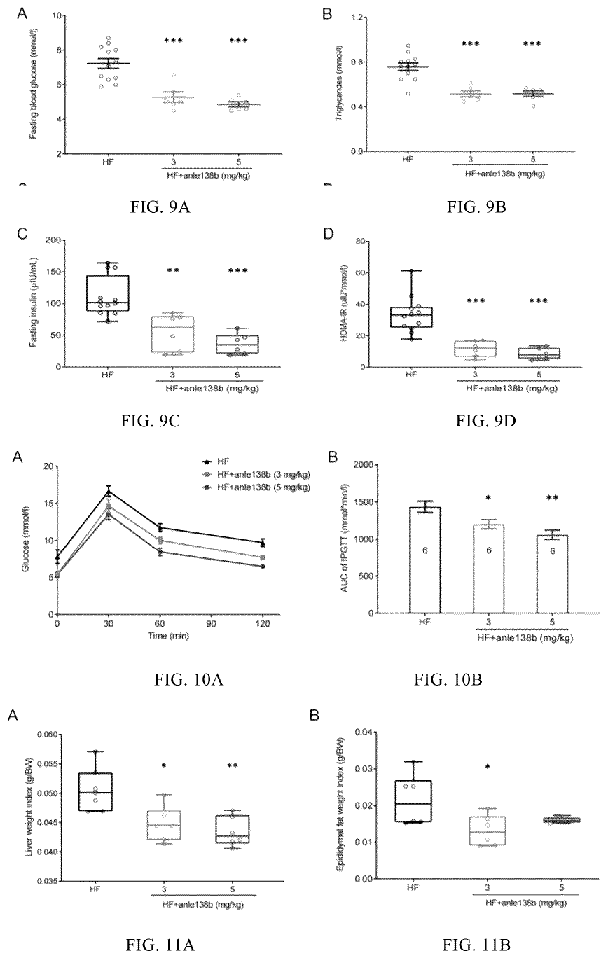

As shown in Table 4 and FIGS. 9A-9D, the fasting blood glucose in the high-fat model group was (7.225±0.99) mmol/l, and the fasting blood glucose after intervention with Anle138b 3 mg/kg and 5 mg/kg as decreased to (5.283±0.74) mmol/l and (4.867±0.36) mmol/l, respectively; the fasting insulin was decreased from (110.8±30.99) μIU/mL to (55.24±28.94) μIU/mL and (36.27±16.53) μIU/mL, respectively; the triglyceride was decreased from (0.7571±0.12) mmol/L to (0.5138±0.06) mmol/L and (0.5164±0.06) mmol/L, respectively; the insulin resistance index was decreased from (33.42±11.67) μIU/mL*mmol/L to (11.6±5.044) μIU/mL*mmol/L and (8.446±3.617) μIU/mL*mmol/L, respectively; all the above-mentioned changes caused by Anle138b were statistically different from those before administration. It could be concluded that the Anle138b intervention could effectively improve the hyperinsulinemia induced by high-fat diet, reduce the blood lipids and the blood glucose, improve the insulin resistance, and improve the metabolism of rats.

TABLE 4
Influence of Anle138b Intervention on the Metabolic Indexes of
High-fat-induced Rat insulin resistance models

| | | | | | Insulin Resistance |
|---|---|---|---|---|---|
| | | Fasting Blood | Fasting Insulin | Triglyceride | Index |
| Group | n | Glucose mmol/L | μIU/mL | mmol/L | μIU/mL*mmol/L |
| Model Control Group | 12 | 7.225 ± 0.99 | 110.8 ± 30.99 | 0.7571 ± 0.12 | 33.42 ± 11.67 |
| Anle138b 3 | 6 | 5.283 ± 0.74 | 55.24 ± 28.94** | 0.5138 ± 0.06** | 11.6 ± 5.04*** |
| mg/kg/d | | *** | * | | |
| Anle138b 5 | 6 | 4.867 ± 0.36 | 36.27 ± 16.53** | 0.5164 ± 0.06** | 8.446 ± 3.62*** |
| mg/kg/d | | *** | * | * | |

Compared with the model control group, **P < 0.01, ***P < 0.001.

The glucose tolerance test was performed, and the results are as shown in FIGS. 10A-10B. It could be seen that the glucose rate of the rats in the drug group was increased as compared with that of the model group. The area under curve (AUC) was further calculated, and the AUC of the model group was (1,435±187.2) min*mmol/L; the AUC after intervention with Anle138b 3 mg/kg was (1,203±155.6) min*mmol/L, P<0.05; the AUC after intervention with Anle138b 5 mg/kg was (1,059±153.2) min*mmol/L, and there was statistical significance for the differences (P<0.01). This indicated by decreasing the fasting blood glucose, improving hyperinsulinemia and improving the glucose tolerance response that the drug could effectively improve diet-induced insulin resistance in the body.

The liver weight index of the model group was 0.05058±0.004 (n=7); the liver weight index decreased to 0.04475±0.003 after intervention with Anle138b 3 mg/kg; the liver weight index decreased to 0.04344±0.0026 after intervention with Anle138b 5 mg/kg; there was statistical significance for the differences. The fat weight index of the model group was 0.02148±0.00702 (n=6); the fat weight index decreased to 0.01317±0.004212 after intervention with Anle138b 3 mg/kg; the fat weight index decreased to after intervention with Anle138b 5 mg/kg. This indicated that the drug could effectively relieve and reduce the weight of liver and fat after application, as shown in FIGS. 11A-11B.

The results of the pathological examination showed that after 12 weeks of high-fat feeding, unclear demarcation and structural disorder of hepatic lobules of the rats could be observed, there was obvious fat infiltration merging into circular lipid droplets of different sizes in the liver, the central hepatic vein has abnormal structure, accompanied by the infiltration of inflammatory cells and the disordered arrangement of hepatic cord; after intervention with Anle138b 3 mg/kg and 5 mg/kg, respectively, the hepatic steatosis was significantly alleviated, no large lipid droplets were seen, and the central vein structure and hepatic cord arrangement structure recovered, and the infiltration of inflammatory cells was reduced; this indicated that the drug application could effectively alleviate the hepatocyte injury induced by high-fat diet.

Different from the pathological examination of pancreas after 8 weeks of high-fructose feeding, disordered arrangement of pancreas islet cells, enlarged capillaries in the islets, damage to the normal structure of pancreatic acinar cells, locally digested acinar cells and obvious acinar steatosis could be observed after 12 weeks of high-fat feeding; after intervention with Anle138b 3 mg/kg and 5 mg/kg, it could be seen that as the dose increases, the acinar steatosis was significantly alleviated, the structure was restored as compared with that in the model group, and the capillaries in the islet cells were reduced; this indicated that the drug application could effectively alleviate the pancreatic cell damage induced by high-fat diet.

The pathological examination of the epididymal adipose tissue showed that in the model group, the fat structure was disordered, the coronal structure formed by aggregated inflammatory cells was observed in the fat space and obviously increased as compared with that in the high-fructose feeding group, a part formed into a cord shape along the cells, the fat cells were enlarged in different sizes, the normal structure was destroyed; after 2 weeks of drug treatment with Anle138b, it could be seen that with the increase of the drug concentration, the adipocytes were reduced in sizes and arranged regularly, and the tubular structure was reduced.

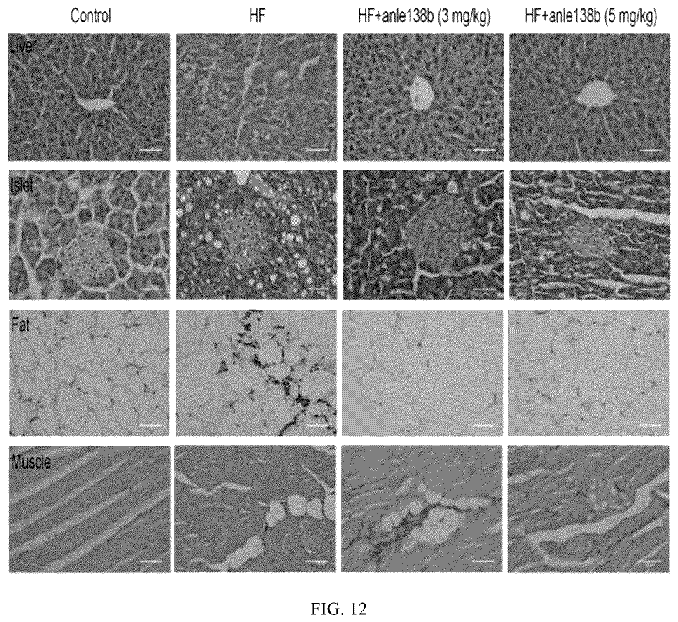

During the 12 weeks of high-fat diet feeding, intramuscular fat infiltration of skeletal muscle cells was observed, intermuscular fat manifestations occurred, and meanwhile, the disordered arrangement of muscle cords and the infiltration of inflammatory cells also occurred; after the drug intervention for 2 weeks, the intermuscular steatosis was relieved, but the arrangement structure of muscle cords did not improve significantly, and there were also manifestations of inflammatory infiltration, as shown in FIG. 12.

### EXAMPLE 3

### Preventive Effect of Anle138b on High-Fat-Induced Rat Insulin Resistance Models

Rats fed with high-fat diet for 5 weeks were randomly divided into high-fat feeding control group (n=6) and drug intervention group with Anle138b 5 mg/kg, and the rats were administered for 3 weeks continuously, once every two days.

The preventive administration with Anle138b 5 mg/kg could reduce the food intake of rats, inhibit the body weight gain, effectively inhibit the increase of fasting blood glucose, insulin, triglyceride and cholesterol, improve the insulin resistance, and improve the glucose tolerance response. In pathological detection, Anle138b could effectively alleviate the tissue structure damage and inflammatory response to pancreas and visceral fat induced by high-fat diet. This indicated that Anle138b had preventive effect on insulin resistance induced by high-fat diet in the rat models.

### (1) Influence of Prevention by Anle138b on the Food Intake and Body Weights of High-Fat-Induced Rat Insulin Resistance Model

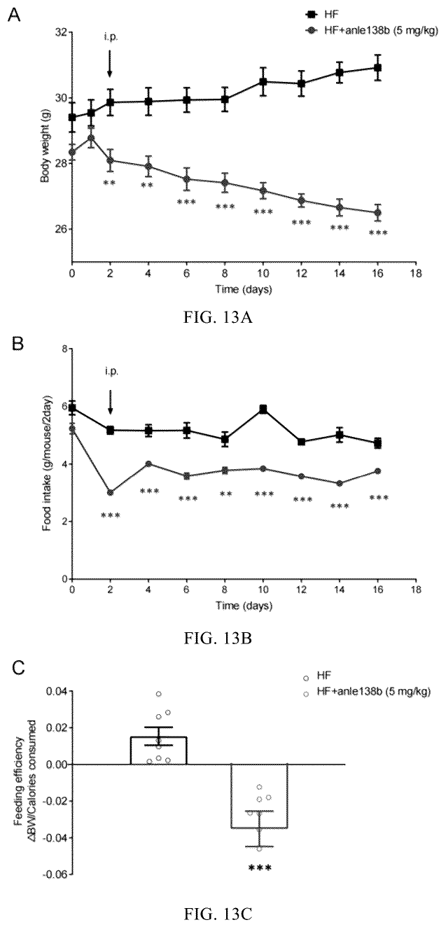

In the prevention experiment, the changes in the food intake and body weight of rats were monitored. As shown in FIGS. 13A-13C, after THE drug intervention with Anle138b, the food intake could be reduced immediately, and at the same time, Anle138b could resist the body weight gain induced by high-fat diet, and the body weight showed a declining trend. At the end point of the prevention experiment, the body weight after drug administration decreased from (30.67 g±0.78) g to (25.8±0.67) g (n=10), P<0.001; the food intake decreased from (2.582±0.27) g/rat to (1.805±0.19) g/rat (n=10), P<0.001; the daily feeding efficiency was calculated, the feeding efficiency of the model group was 0.01534±0.014, and the feeding efficiency of the Anle138b 5 mg/kg group was −0.03518±0.02726, showing a negative increase. It could be seen that in addition to the therapeutic effect on reducing body weight and food intake in high-fat fed rats, Anle138b also had a preventive effect.

### (2) Influence of Prevention by Anle138b on Metabolic Indexes of High-Fat-Induced Rat Insulin Resistance Model

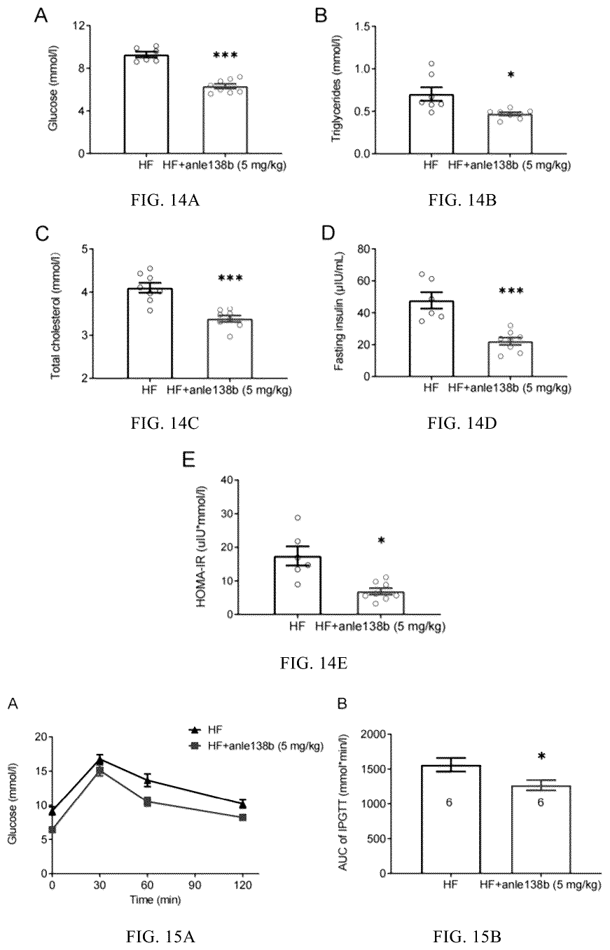

As shown in Table 5, the fasting blood glucose of the model group was (9.283±0.66) mmol/L (n=6), the fasting blood glucose of the drug group was (6.313±0.63) mmol/L (n=8), which was significantly lower than that of the model group, and there was statistical significance for the difference (P<0.001); the triglyceride and the total cholesterol in the model group were (0.7024±0.21) mmol/L and (4.099±0.32) mmol/L, respectively; the triglyceride and the total cholesterol in the drug group decreased to (0.4687±0.05) mmol/L and (3.383±0.21) mmol/L; the fasting serum insulin of the model group was (47.67±12.61) μIU/mL, and the fasting serum insulin drug group decreased to (22.09±6.49) μIU/mL; the insulin resistance index was calculated, the insulin resistance index was (17.43±7.00) μIU/mL*mmol/L in the model group and (6.880±2.73) μIU/mL*mmol/L in the drug group, as shown in FIGS. 14A-14E. It could be seen that Anle138b 5 mg/kg could effectively prevent metabolic disorders caused by high-fat diet.

TABLE 5
Influence of Prevention by Anle138b on Metabolic Indexes
of High-fat-induced Rat insulin resistance model

| | | Fasting | | | | Insulin |
|---|---|---|---|---|---|---|
| | | Blood | Fasting | | Total | Resistance |
| | | Glucose | Insulin | Triglyceride | Cholesterol | Index |
| Group | n | mmol/L | μIU/mL | mmol/L | mmol/L | μIU/mL*mmol/L |
| Model | 6 | 9.283 ± 0.66 | 47.67 ± 12.61 | 0.7024 ± 0.21 | 4.099 ± 0.32 | 17.43 ± 7.00 |
| Control | | | | | | |
| Group | | | | | | |
| Anle138b | 8 | 6.313 ± 0.63*** | 22.09 ± 6.49*** | 0.4687 ± 0.05* | 3.383 ± 0.21*** | 6.880 ± 2.73* |
| 5 mg/kg/d | | | | | | |

Compared with the model control group, **P < 0.05, ***P < 0.001.

The results of the glucose tolerance test are shown in FIGS. 15A-15B. Anle138b 5 mg/kg could effectively improve the impaired glucose tolerance induced by high-load diet. The area under curve (AUC) was calculated, the AUC of the model group was (1,562±97) min*mmol/L (n=6); the AUC of the drug group was (1,269±74) min*mmol/L (n=6), which was significantly lower than that of the model group, and there was statistical significance for the difference (P<0.05).

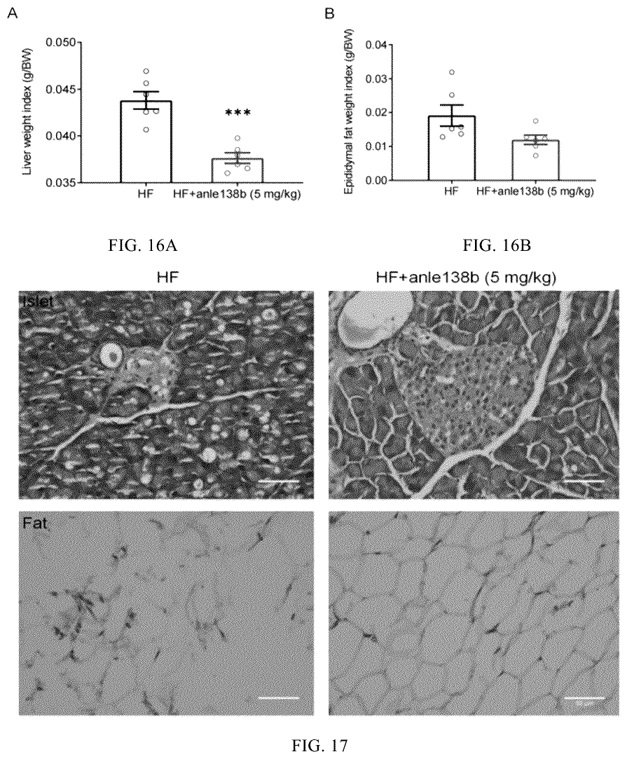

As shown in FIGS. 16A-16B, in the prevention experiment, the liver weight and the fat weight can also be effectively reduced, wherein the liver weight index decreased from 0.04381±0.002 in the model group to 0.03763±0.001 in the drug group, P<0.001; the fat weight index decreased from 0.0191±0.008 in the model group to 0.01195±0.003 in the drug group, and the fat weight index showed a trend of significant decline.

The pathological examination showed that as shown in FIG. 17, the preventive administration with Anle138b 5 mg/kg could significantly inhibit the steatosis of pancreatic acinar cells, and intraislet vasodilation and cell microcystic changes could still be observed, but the degree was relieved as compared with that of the model group; similarly, the drug preventive intervention relieved the structural disorder of the adipose tissue, restored the normal structure and size of adipocytes, and reduced the changes in the inflammatory response of the adipose tissue. It was concluded that the drug Anle138b had a preventive effect on the pathological changes induced by high-fat diet.

### EXAMPLE 3

### Influence of Intervention with Anle138b and Paired-Feed Group on Rat Insulin Resistance Models

The rats fed with high-fat diet for 8 weeks were divided into paired-feed group (n=7) (the food intake was the same as that of high-fat Anle138b 5 mg/kg/d) and control group continuously fed with high-fat diet.

There was no significant difference in the changes in the body weight measurement between the pair-feed group and the Anle138b 5 mg/kg group, indicating that the drug intervention induced body weight loss was caused by the decrease in the food intake. There was no decrease in the serum fasting blood glucose, triglyceride and insulin concentrations in the paired-feed group, indicating that simply reducing the food intake could not inhibit the glucose metabolism disorder and hyperinsulinemia caused by high-fat diet, also indicating that the effect of Anle138b on improving the insulin resistance was independent of suppressing the dietary decline. The comparison of the pathological examination showed that the paired-feed group could neither reduce the ectopic fat storage in liver and muscle, nor improve the vascular proliferation, structural disorder and acinar steatosis in pancreatic islets. In summary, Anle138b could alleviate the hepatic and pancreatic steatosis in rat insulin resistance models, and this function was independent of simply reducing the food intake; only its weight-reducing effect was associated with the reduction in the food intake.

Pair-feed (PF) group: the pair-feed group was set according to the daily food intake in the high-fat group after intervention with Anle138b 5 mg/kg, the rats were given the same amount of high-fat diet every day, and the changes in the body weight and the metabolic indexes were measured.

### (1) Influence of Anle138b Treatment+PF Group on the Food Intake and Body Weights of High-Fat-Induced Rat Insulin Resistance Models

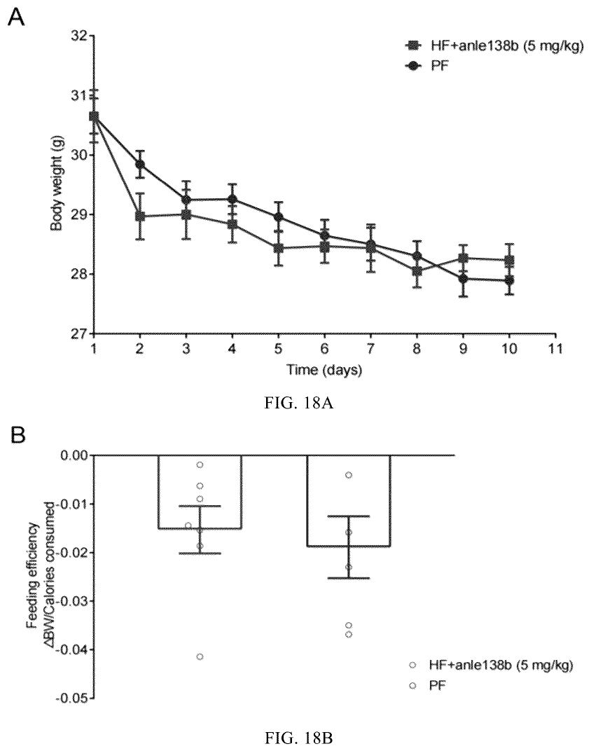

As shown in FIGS. 18A-18B, in the PF group, the body weight was also reduced after reducing the food intake, the average body weight of (28.23±0.66) g and the feeding efficiency of −0.015±0.013 (n=6) after intervention with Anle138b 5 mg/kg were not statistically different from the body weight of (27.94±0.76) g and the feeding efficiency of −0.019±0.016 (n=7) in the PF group; this indicated that the body weight loss after the drug intervention with Anle138b was caused by the reduction in food intake.

### (2) Influence of Anle138b Treatment+PF Group on Metabolic Indexes of Rat Insulin Resistance Models

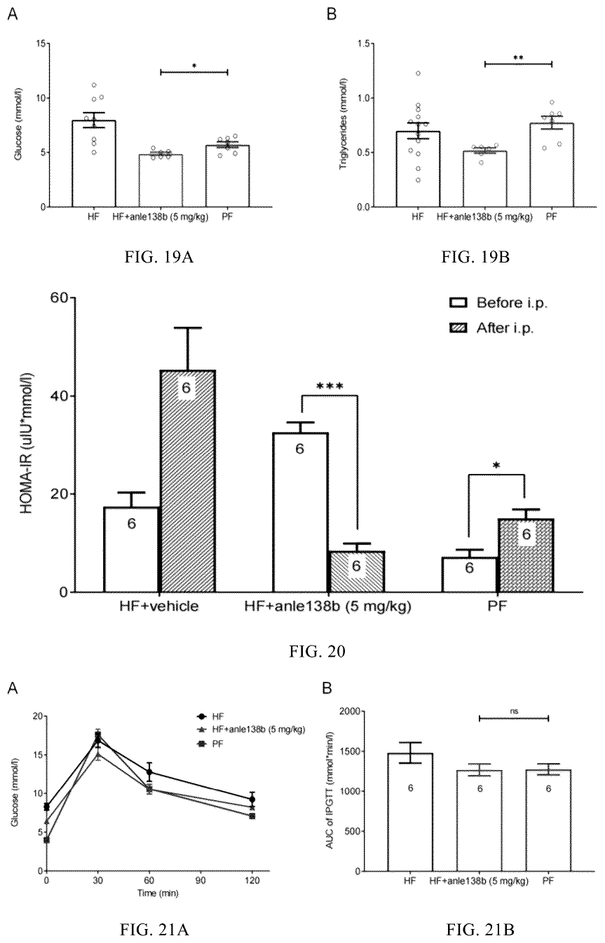

The influence of Anle138b treatment+PF group on the metabolic indexes of rat insulin resistance models is as shown in FIGS. 19A-19B, the fasting blood glucose of the PF group was reduced to (5.7±0.71) mmol/L, and there was statistical significance for the difference (P<0.05). After intervention with Anle138b 5 mg/kg, the blood glucose level was further reduced to (4.87±0.36) mmol/L, which was statistically different from that in the PF group (P<0.05). This indicated that the control of food intake could reduce the blood glucose level of the HF model rats, but Anle138b reduced the blood glucose level of the HF model rats to a lower level than that in the control group, suggesting that mechanisms other than food control were involved. The triglyceride level in the PF group was (0.77±0.16) mmol/L, and there was no significant difference between the two groups (P>0.05), indicating that simply controlling the food intake could not reduce the triglyceride level of the HF model rats. However, the triglyceride was reduced to (0.52±0.06) mmol/L after drug intervention with Anle138b 5 mg/kg, and there was statistical significance for the difference (P<0.01).

The comparison of the insulin resistance is as shown in FIG. 20. By the self control after intervention in the PF group, the insulin resistance value was increased, and simply reducing food intake did not improve the insulin resistance. The indexes of the metabolic assay showed that although the paired feeding could reduce the body weight and the blood glucose, but the paired feeding did not improve the high-fat-diet-induced hyperlipidemia and insulin resistance.

As shown in FIGS. 21A-21B, by the self control before and after intervention in the PF group, the AUC decreased, and the AUC was not significantly different from that of intervention with Anle138b 5 mg/kg; however, the blood glucose detection at 30 min showed that the intervention failed to effectively stimulate the insulin secretion to rapidly lower the blood glucose in the PF group, and it could still be seen that the glucose response curve shifted to the right by curve analysis. The glucose tolerance test showed that the dietary reduction could effectively improve the high-fat-induced impaired glucose tolerance.

As shown in FIGS. 22A-22B, there was no significant difference in the liver weight index and fat weight index between the PF group and the drug intervention group with Anle138b, indicating that the dietary reduction could effectively inhibit the increase in the organ weight.

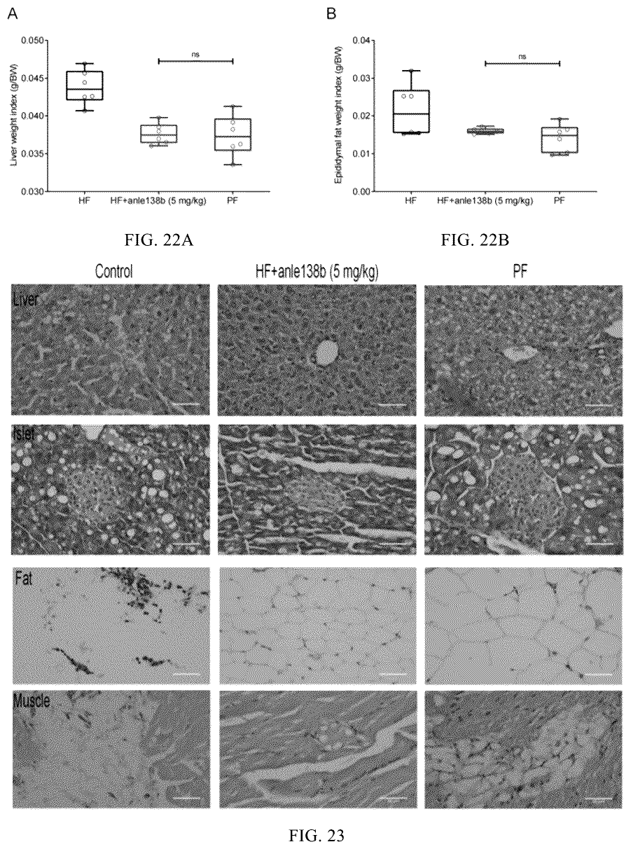

The pathological examination showed that, as shown in FIG. 23, the PF group could effectively improve the inflammatory response of adipocytes and the size and structure of adipocytes; however, compared with the drug intervention group, the pancreas still had obvious acinar steatosis, islet structure disorder and capillary hyperplasia; the liver tissue with structural disorder still had obvious fat infiltration and inflammatory reaction; the pathological examination of skeletal muscle showed that the fat infiltration also increased as compared with that of the drug intervention group, and the obvious inflammatory reaction was not significantly improved as compared with that of the model group. The pathological examination showed that the simple reduction of high-fat diet could partially alleviate the steatosis, but did not have the effect on improving the hepatic, pancreatic and muscular steatosis, indicating that the effect of Anle138b on improving the hepatic, pancreatic and muscular steatosis was independent of the reduction of food intake.

Therefore, the application of Anle138b in a drug for improving diet-induced insulin resistance is adopted in the present invention, Anle138b has obvious improvement effects on both preventing and treating insulin resistance, and can reduce the intake efficiency and the body weight. Besides, Anle138b can effectively improve diet-induced ectopic fat storage and adipose tissue hypertrophy, and the effect of Anle138b on improving the insulin resistance is independent of the reduction of food intake.

Finally, it shall be stated that the embodiments above are only used for explaining, rather than restricting, the technical schemes of the present invention. Although the present invention is explained in detail referring to the preferred embodiments, those of skill in the art should be understood that, the technical schemes of the present invention can be modified or equivalently replaced, whereas the modifications or equivalent replacements will not make the modified technical scheme deviate from the spirit and scope of the technical schemes of the present invention.

1. A method of an application of Anle138b in a drug for improving a diet-induced insulin resistance.

2. The method according to claim 1, wherein an improvement of the diet-induced insulin resistance comprises an improvement of an insulin resistance through a prevention or a treatment.

3. The method according to claim 1, wherein a reduction of a diet-induced glucose increase is achieved through an improvement of the diet-induced insulin resistance.

4. A method of an application of Anle138b in a drug for reducing a food intake and a body weight of patients with insulin resistance diseases.

5. A method of an application of Anle138b in a drug for reducing a blood lipid of patients with insulin resistance diseases.
